SEQUENCE LISTING (33 sequences)

SEQ ID NO: 1 - protein, 11 aa, Artificial Sequence
  Amino acid sequence generated by GATEWAY (Registered) plasmids
CDPAFLYKVVD

SEQ ID NO: 2 - protein, 10 aa, Artificial Sequence
  Amino acid sequence generated by GATEWAY (Registered) plasmids
CDPAFLYKVV

SEQ ID NO: 3 - protein, 10 aa, Artificial Sequence
  Amino acid sequence geneated by GATEWAY (Registered) plasmids
DPAFLYKVVD

SEQ ID NO: 4 - protein, 9 aa, Artificial Sequence
  Amino acid sequence generated by GATEWAY (Registered) plasmid
CDPAFLYKV

SEQ ID NO: 5 - protein, 9 aa, Artificial Sequence
  Amino acid sequence generated by GATEWAY (Registered) plasmid
DPAFLYKVV

SEQ ID NO: 6 - protein, 9 aa, Artificial Sequence
  Amino acid sequence generated by GATEWAY (Registered) plasmid
PAFLYKVVD

SEQ ID NO: 7 - protein, 8 aa, Artificial Sequence
  Amino acid sequence generated by GATEWAY (Registered) plasmid
CDPAFLYK

SEQ ID NO: 8 - protein, 8 aa, Artificial Sequence
  Amino acid sequence generated by GATEWAY (Registered) plasmid
DPAFLYKV

SEQ ID NO: 9 - protein, 8 aa, Artificial Sequence
  Amino acid sequence generated by GATEWAY (Registered) plasmid
PAFLYKVV

SEQ ID NO: 10 - protein, 7 aa, Artificial Sequence
  Amino acid sequence generated by GATEWAY (Registered) plasmid
FLYKVVD

SEQ ID NO: 11 - protein, 7 aa, Artificial Sequence
  Amino acid sequence generated by GATEWAY (Registered) plasmid
CDPAFLY

SEQ ID NO: 12 - protein, 7 aa, Artificial Sequence
  Amino acid sequence generated by GATEWAY (Registered) plasmid
DPAFLYK

SEQ ID NO: 13 - protein, 7 aa, Artificial Sequence
  Amino acid sequence generated by GATEWAY (Registered) plasmid
PAFLYKV

SEQ ID NO: 14 - protein, 7 aa, Artificial Sequence
  Amino acid sequence generated by GATEWAY (Registered) plasmid
AFLYKVV

SEQ ID NO: 15 - protein, 7 aa, Artificial Sequence
  Amino acid sequence generated by GATEWAY (Registered) plasmid
FLYKVVD

SEQ ID NO: 16 - protein, 6 aa, Artificial Sequence
  Amino acid sequence generated by GATEWAY (Registered) plasmid
CDPAFL

SEQ ID NO: 17 - protein, 6 aa, Artificial Sequence
  Amino acid sequence generated by GATEWAY (Registered) plasmid
DPAFLY

SEQ ID NO: 18 - protein, 6 aa, Artificial Sequence
  Amino acid sequence generated by GATEWAY (Registered) plasmid
PAFLYK

SEQ ID NO: 19 - protein, 6 aa, Artificial Sequence
  Amino acid sequence generated by GATEWAY (Registered) plasmid
AFLYKV

SEQ ID NO: 20 - protein, 6 aa, Artificial Sequence
  Amino acid sequence generated by GATEWAY (Registered) plasmid
FLYKVV

SEQ ID NO: 21 - protein, 6 aa, Artificial Sequence
  Amino acid sequence generated by GATEWAY (Registered) plasmid
LYKVVD

SEQ ID NO: 22 - protein, 5 aa, Artificial Sequence
  Amino acid sequence generated by GATEWAY (Registered) plasmid
CDPAF

SEQ ID NO: 23 - protein, 5 aa, Artificial Sequence
  Amino acid sequence generated by GATEWAY (Registered) plasmid
DPAFL

SEQ ID NO: 24 - protein, 5 aa, Artificial Sequence
  Amino acid sequence generated by GATEWAY (Registered) plasmid
PAFLY

SEQ ID NO: 25 - protein, 5 aa, Artificial Sequence
  Amino acid sequence generated by GATEWAY (Registered) plasmid
AFLYK

SEQ ID NO: 26 - protein, 5 aa, Artificial Sequence
  Amino acid sequence generated by GATEWAY (Registered) plasmid
FLYKV

SEQ ID NO: 27 - protein, 5 aa, Artificial Sequence
  Amino acid sequence generated by GATEWAY (Registered) plasmid
LYKVV

SEQ ID NO: 28 - protein, 5 aa, Artificial Sequence
  Amino acid sequence generated by GATEWAY (Registered) plasmid
YKVVD

SEQ ID NO: 29 - DNA, 47 bp, Artificial Sequence
  Synthetic primer for the insertion of a SphI restriction site in the yeast shuttle vector p426
cgaaattgttcctacgagctcgcatgcttttgttccctttagtgagg

SEQ ID NO: 30 - DNA, 47 bp, Artificial Sequence
  Synthetic primer for the insertion of a SphI restriction site in the yeast shuttle vector p426
gactcactatagggcgaattggcatgcggccgcaaattaaagccttc

SEQ ID NO: 31 - DNA, 47 bp, Artificial Sequence
  Synthetic primer for the insertion of a NotI restriction site between PHXT7 and TCYC1 in the yeast shuttle vector p426
cataactaattacatgactcgagcggccgcccgggggatccactaga

SEQ ID NO: 32 - DNA, 30 bp, Artificial Sequence
  Synthetic primer for the amplification 4CL5 gene from Arabidopsis thaliana
gcggccgcatggtgctccaacaacaaacgc

SEQ ID NO: 33 - DNA, 30 bp, Artificial Sequence
  Synthetic primer for the amplification 4CL5 gene from Arabidopsis thaliana
gcggccgcctatttagagcacatggtttcc

(19) United States
(12) Patent Application Publication
(10) Pub. No.: US 2012/0202244 A1
(43) Pub. Date: Aug. 9, 2012

(54) Antibodies Capable of Specifically Binding to a Specific Amino Acid Sequence

(75) Inventors: Dominique Loque, Albany, CA (US); Wolf B. Frommer, San Francisco, CA (US)

(73) Assignees: Carnegie Institution of Washington, Washington, DC (US); The Regents of the University of California, Oakland, CA (US)

(21) Appl. No.: 13/273,971

(22) Filed: Oct. 14, 2011

Related U.S. Application Data
(60) Provisional application No. 61/393,827, filed on Oct. 15, 2010.

(51) Int. Cl. C12P 21/02; C12N 1/19; C12N 5/10; C07K 2/00; C07K 16/00; C12N 15/13; C07K 1/14; C12N 15/63; C12N 5/12

(52) U.S. Cl. 435/69.1; 435/320.1; 435/254.21; 435/252.3; 435/252.31; 435/252.33; 435/419; 435/331; 530/387.9; 536/23.53; 530/344

(57) ABSTRACT

The present invention provides for an antibody or fragment thereof capable of specifically binding to an epitope of the amino acid sequence CDPAFLYKVVD (SEQ ID NO:1) or a fragment of at least 5, 6, or 7 amino acids thereof.

### CROSS-REFERENCE TO RELATED APPLICATIONS

This application claims priority to U.S. Provisional Patent Application Ser. No. 61/393,827, filed Oct. 15, 2010, which is hereby incorporated by reference in its entirety.

### STATEMENT OF GOVERNMENTAL SUPPORT

The invention was made with government support under Contract No. DE-AC02-05CH11231 awarded by the U.S. Department of Energy under. The government has certain rights in the invention.

### FIELD OF THE INVENTION

The present invention is in the field of antibodies.

### SUMMARY OF THE INVENTION

The present invention provides for an antibody or fragment thereof capable of specifically binding to an epitope of the amino acid sequence CDPAFLYKVVD (SEQ ID NO:1) or a fragment of at least 5, 6, or 7 amino acids thereof.

The epitope can be one presented by an amino acid sequence selected from the group consisting of CDPAFLYKVV (SEQ ID NO:2), DPAFLYKVVD (SEQ ID NO:3), CDPAFLYKV (SEQ ID NO:4), DPAFLYKVV (SEQ ID NO:5), PAFLYKVVD (SEQ ID NO:6), CDPAFLYK (SEQ ID NO:7), DPAFLYKV (SEQ ID NO:8), PAFLYKVV (SEQ ID NO:9), FLYKVVD (SEQ ID NO:10), CDPAFLY (SEQ ID NO:11), DPAFLYK (SEQ ID NO:12), PAFLYKV (SEQ ID NO:13), AFLYKVV (SEQ ID NO:14), FLYKVVD (SEQ ID NO:15), CDPAFL (SEQ ID NO:16), DPAFLY (SEQ ID NO:17), PAFLYK (SEQ ID NO:18), AFLYKV (SEQ ID NO:19), FLYKVV (SEQ ID NO:20), LYKVVD (SEQ ID NO:21), CDPAF (SEQ ID NO:22), DPAFL (SEQ ID NO:23), PAFLY (SEQ ID NO:24), AFLYK (SEQ ID NO:25), FLYKV (SEQ ID NO:26), LYKVV (SEQ ID NO:27), and YKVVD (SEQ ID NO:28).

The present invention relates to a polynucleotide encoding the antibody or fragment thereof of the present invention, vectors comprising said polynucleotide as well as cells comprising the afore-mentioned polynucleotide or vector. The present invention also provides a method for preparing antibodies capable of binding to an epitope of the amino acid sequence CDPAFLYKVVD (SEQ ID NO:1).

The present invention provides for a hybridoma capable of producing an antibody or fragment thereof of the present invention.

The present invention provides for a method of isolating a peptide of interest, comprising: (a) contacting (i) a peptide of interest linked to the amino acid sequence CDPAFLYKVVD (SEQ ID NO:1) or a fragment thereof, and (ii) the antibody or fragment thereof of the present invention, and (b) separating at least a partial population of the antibody or fragment thereof, and any bound molecule thereto, from molecules not bound to the antibody or fragment thereof.

In some embodiments of the invention, the contacting step comprises introducing a first solution comprising the peptide of interest linked to the amino acid sequence CDPAFLYKVVD (SEQ ID NO:1) or a fragment thereof, and a second solution comprising the antibody or fragment thereof. In some embodiments of the invention, the method further comprises linking the peptide of interest to the amino acid sequence CDPAFLYKVVD (SEQ ID NO:1) or a fragment thereof.

In some embodiments of the invention, the method further comprises expressing the peptide of interest linked to the amino acid sequence CDPAFLYKVVD (SEQ ID NO:1) or a fragment thereof in a host cell, or in vitro in a reaction solution, comprising a polynucleotide encoding peptide of interest linked to the amino acid sequence CDPAFLYKVVD (SEQ ID NO:1) or a fragment thereof. In some embodiments of the invention, the method further comprises linking a first polynucleotide encoding the peptide of interest and a second polynucleotide encoding the amino acid sequence CDPAFLYKVVD (SEQ ID NO:1) or a fragment thereof. The linking of the first polynucleotide encoding the peptide of interest and the second polynucleotide can comprise linking the second polynucleotide to the 5′ end of, 3′ end of, or within the first polynucleotide.

The present invention provides for a kit comprising: a vector comprising a nucleotide sequence encoding the amino acid sequence CDPAFLYKVVD (SEQ ID NO:1) or a fragment thereof linked to one or more restriction sites, and an antibody or fragment thereof capable of specifically binding to an epitope of the amino acid sequence CDPAFLYKVVD (SEQ ID NO:1) or a fragment thereof. When an open reading frame of a peptide of interest is inserted within one of the one or more restriction sites, the vector is capable of expressing a hybrid polypeptide comprisn the amino acid sequence CDPAFLYKVVD (SEQ ID NO:1) or a fragment thereof linked of the peptide of interest. The expression of the hybrid polypeptide can take place in vitro or in vivo, in a suitable host cell. By assaying for the level of the amino acid sequence CDPAFLYKVVD (SEQ ID NO:1) or a fragment thereof present, or binding the amino acid sequence CDPAFLYKVVD (SEQ ID NO:1) or a fragment thereof present, using antibody or fragment thereof of the present invention, one can assay the amount of the peptide of interest and/or isolate or purify the peptide of interest.

Suitable vectors for use in the method include the following commercially available vectors: GATEWAY® vectors (commercially available from Invitrogen Corp., Carlsbad, Calif.), such as pcDNA™-DEST40, pBAD-DEST49 Gateway®, pcDNA™ 6.2/GFP-DEST, pcDNA™ 6.2/GFP-GW/p64^{TAG}, pcDNA™ 6.2/V5-DEST, pcDNA™ 6.2/V5-GW/p64^{TAG}, and the like. Such vectors are described in the following Invitrogen Corp. publications: “Gateway® pcDNA™-DEST40 Vector” (Cat. no. 12274-015, Jul. 2, 2008), “pBAD-DEST49 Gateway® Destination Vector” (Cat. no. 12283-016, Ver. E, Jul. 21, 2008), and “pcDNA-DEST40 Gateway™ Vector” (Cat. no. 12274-015, Ver. C, Aug. 13, 2002) (all of which are herein incorporated by reference).

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the expression analysis of hydroxycinnamoyl/benzoyl-CoA:anthranilate N-hydroxycinnamoyl/benzoyltransferase (HCBT), an enzyme from Dianthus caryophyllus, which has affinity for anthranilate and p-coumaroyl-CoA and is capable of producing N-(4′-hydroxycinnamoyl)-anthranilate in vitro. Recombinant yeast cells grown to an OD₆₀₀=1 are harvested by centrifugation for protein extraction, and 5 μg of soluble protein are analyzed using immunobloting techniques. For protein extracts obtained from cells harboring the pDRf1-4CL5-HCBT or pDRf1-HCBT vectors, recombinant tagged HCBT is detected around 53 kDa using the universal antibody and according to the position of known markers. Protein extracts from yeast cells harboring the pDRf1-4CL5-GW or pDRfl empty vectors are also analyzed as negative controls.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “TAG” as used herein refers to the amino acid sequence CDPAFLYKVVD (SEQ ID NO:1) or a fragment of at least 5,6, or 7 amino acids thereof, including the amino acid sequences represented by SEQ ID NOs:2-28.

The terms “host cell” and “host microorganism” are used interchangeably herein to refer to a living biological cell that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host microorganism, wherein the expression vector contains a nucleic acid sequence coding for an enzyme that is not normally found in a host microorganism. With reference to the host microorganism's genome, then, the nucleic acid sequence that codes for the enzyme is heterologous.

The terms “expression vector” or “vector” refer to a compound and/or composition that transduces, transforms, or infects a host microorganism, thereby causing the cell to express nucleic acids and/or proteins other than those native to the cell, or in a manner not native to the cell, or that causes in vitro transcription. An “expression vector” contains a sequence of nucleic acids (ordinarily RNA or DNA) to be expressed by the host microorganism. Optionally, the expression vector can also comprise material(s) to aid in achieving entry of the nucleic acid into the host microorganism, such as a virus, liposome, protein coating, or the like. The expression vectors can include those into which a nucleic acid sequence can be inserted, along with any required operational elements. Optionally, the expression vector can be one that can be transferred into a host microorganism and replicated therein. In some embodiments, the expression vectors are plasmids, including those with restriction sites that have been well documented and that contain the operational elements required for transcription of the nucleic acid sequence. Such plasmids, as well as other expression vectors, are well known to those of ordinary skill in the art.

The term “transduce” as used herein refers to the transfer of a sequence of nucleic acids into a host microorganism or cell. Only when the sequence of nucleic acids becomes stably replicated by the cell does the host microorganism or cell become “stably transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce. “Transformation” can also be transient. For example, a sequence of nucleic acids, such as DNA or RNA, can be transferred into a host microorganism or cell wherein expression from the sequence of nucleic acids takes place while the sequence of nucleic acids is not replicable or does not replicate.

The terms “isolated” or “biologically pure” refer to material that is substantially or essentially free of components that normally accompany it in its native state.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing normucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

The antibody or fragment thereof of the present invention comprises at least one (or 2, 3, 4, 5, or 6) complementarity determining region (CDR) of the V_{H} and/or V_{L} region of an antibody or fragment thereof comprising the amino acid sequence that specifically recognizes the TAG. Alternatively, and/or in addition the antibody of the invention comprises at least 1, 2 or 3 CDR(s) of the V_{L} region of an immunoglobulin chain that binds to the TAG.

The person skilled in the art knew that each variable domain (the heavy chain V_{H} and light chain V_{L}) of an antibody comprises three hypervariable regions, sometimes called complementarity determining regions or “CDRs” flanked by four relatively conserved framework regions or “FRs”. The CDRs contained in the variable regions of the antibody of the invention can be determined, e.g., according to Kabat, Sequences of Proteins of Immunological Interest (U.S. Department of Health and Human Services, third edition, 1983, fourth edition, 1987, fifth edition 1990). The person skilled in the art will readily appreciate that the variable domain of the antibody having the above-described variable domain can be used for the construction of other polypeptides or antibodies of desired specificity and biological function. Thus, the present invention also encompasses polypeptides and antibodies comprising at least one CDR of the above-described variable domain and which advantageously has substantially the same or similar binding properties as the antibody described in the appended examples. The person skilled in the art will readily appreciate that using the variable domains or CDRs described above antibodies can be constructed according to methods known in the art, e.g., as described in EP-A10 451 216 and EP-A10 549 581.

In accordance with the present invention a screening assay that specifically allows the detection of anti-TAG antibodies capable of recognizing the TAG directly expressed in cells without the requirement of antigen purification can be chosen to identify and purify antibodies directed at conformation-dependent determinants. The assay was also based on expression of a genotype 1a derived antigen thus allowing for the characterization of cross-reactive anti-TAG antibodies and epitopes.

In some embodiments of the invention, said antibody is a monoclonal antibody, a polyclonal antibody, a single chain antibody, or fragment thereof that specifically binds said TAG also including bispecific antibody, synthetic antibody, antibody fragment, such as Fab, Fv or scFv fragments etc., or a chemically modified derivative of any of these. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals with modifications developed by the art. Furthermore, antibodies or fragments thereof to the aforementioned epitopes can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope of the TAG (Schier, Human Antibodies Hybridomas 7 (1996), 97-105; Malmborg, J. Immunol. Methods 183 (1995), 7-13). The production of chimeric antibodies is described, for example, in WO89/09622. As discussed above, the antibody of the invention may exist in a variety of forms besides complete antibodies; including, for example, Fv, Fab and F(ab)₂, as well as in single chains; see e.g. WO88/09344. In case of bispecific antibodies where one specificity is directed to the TAG and the other is directed to another epitope.

The antibodies of the present invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y.

In another embodiment the present invention relates to a polynucleotide encoding at least a variable region of an immunoglobulin chain of any of the before described antibodies of the invention. One form of immunoglobulin constitutes the basic structural unit of an antibody. This form is a tetramer and consists of two identical pairs of immunoglobulin chains, each pair having one light and one heavy chain. In each pair, the light and heavy chain variable regions or domains are together responsible for binding to an antigen, and the constant regions are responsible for the antibody effector functions. In addition to antibodies, immunoglobulins may exist in a variety of other forms (including less than full-length that retain the desired activities), including, for example, Fv, Fab, and F(ab′)2, as well as single chain antibodies (e.g., Huston, Proc. Nat. Acad. Sci. USA 85 (1988), 5879-5883 and Bird, Science 242 (1988), 423-426); see also supra. An immunoglobulin light or heavy chain variable domain consists of a “framework” region interrupted by three hypervariable regions, also called CDR's; see supra.

The antibodies of the present invention can be produced by expressing recombinant DNA segments encoding the heavy and light immunoglobulin chain(s) of the antibody invention either alone or in combination.

The polynucleotide of the invention encoding the above described antibody may be, e.g., DNA, cDNA, RNA or synthetically produced DNA or RNA or a recombinantly produced chimeric nucleic acid molecule comprising any of those polynucleotides either alone or in combination. In some embodiments, the polynucleotide is part of a vector. Such vectors may comprise further genes such as marker genes which allow for the selection of said vector in a suitable host cell and under suitable conditions. In some embodiments, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, such as mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. In this respect, the person skilled in the art will readily appreciate that the polynucleotides encoding at least the variable domain of the light and/or heavy chain may encode the variable domains of both immunoglobulinchains or only one. Likewise, said polynucleotides may be under the control of the same promoter or may be separately controlled for expression. Possible regulatory elements permitting expression in prokaryotic host cells comprise, e.g., the PL, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogen), or pSPORT1 (GIBCO BRL).

In some embodiments, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the immunoglobulin light chains, heavy chains, light/heavy chain dimers or intact antibodies, binding fragments or other immunoglobulin forms may follow; see, Beychok, Cells of Immunoglobulin Synthesis, Academic Press, N.Y., (1979).

As described above, the polynucleotide of the invention can be used alone or as part of a vector to express a peptide of interest in cells, in vitro, or in a cell-free system. The polynucleotides or vectors of the invention are introduced into the cells which in turn produce the antibody. Further, the present invention relates to vectors, particularly plasmids, cosmids, viruses and bacteriophages used conventionally in genetic engineering that comprise a polynucleotide encoding a variable domain of an immunoglobulin chain of an antibody of the invention; optionally in combination with a polynucleotide of the invention that encodes the variable domain of the other immunoglobulin chain of the antibody of the invention. In some embodiments, the vector is an expression vector. Methods which are well known to those skilled in the art can be used to construct recombinant vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989). An example of a cell-free system is the TNT® SP6 High-Yield Wheat Germ Protein Expression System (cell free protein expression) which is based on an optimized wheat germ extract, is a single-tube, coupled transcription/translation system designed to express proteins (commercially available from Promega Corp., Madison, Wis.).

The peptide of interest can be a peptide of any suitable number of amino acids. In some embodiments, the peptide of interest is equal to or less than about 200 amino acid residues in length. In some embodiments, the peptide of interest is equal to or less than about 100 amino acid residues in length. In some embodiments, the peptide of interest is equal to or more than about 200 amino acid residues in length. In some embodiments, the peptide of interest is equal to or more than about 100 amino acid residues in length.

The nucleic acid constructs of the present invention comprise nucleic acid sequences encoding (a) the antibody of the present invention, or (b) the TAG and optionally a peptide of interest. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme or peptide of interest can be incorporated into an expression vector. Incorporation of the individual nucleic acid sequences may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector. The TAG can be linked to the N-terminus, C-terminus, or within the sequence of the peptide of interest.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the LacI repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes, or curing of an auxotrophy.

The polynucleotides and vectors of the invention can be reconstituted into liposomes for delivery to cells. The vectors containing the polynucleotides of the invention (e.g., the heavy and/or light variable domain(s) of the immunoglobulin chains encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

The present invention furthermore relates to host cells transformed with a polynucleotide or vector of the invention. The polynucleotide or vector of the invention which is present in the host cell may either be integrated into the genome of the host cell or it may be maintained extrachromosomally. The host cell can be any prokaryotic or eukaryotic cell, such as a bacterial, insect, fungal, plant, animal or human cell. The fungal cells can be of the genus Saccharomyces, in particular those of the species S. cerevisiae. The term “prokaryotic” is meant to include all bacteria which can be transformed or transfected with a DNA or RNA molecules for the expression of an antibody of the invention or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the antibodies or immunoglobulin chains encoded by the polynucleotide of the present invention may be glycosylated or may be non-glycosylated. Antibodies of the invention or the corresponding immunoglobulin chains may also include an initial methionine amino acid residue. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the antibody of the invention or the corresponding immunoglobulin chains in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic animals, preferably mammals, comprising cells of the invention may be used for the large scale production of the (poly)peptide of the invention.

Thus, in a further embodiment, the present invention relates to a method for the production of an antibody or fragment thereof capable of recognizing the TAG comprising (a) culturing the cell of the invention; and (b) isolating said antibody or functional fragment or immunoglobulin chain(s) thereof from the culture,

The transformed hosts can be grown in fermentors and cultured according to techniques known in the art to achieve optimal cell growth. Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis and the like; see, Scopes, “Protein Purification”, Springer-Verlag, N.Y. (1982). The antibody or its corresponding immunoglobulin chain(s) of the invention can then be isolated from the growth medium, cellular lysates, or cellular membrane fractions. The isolation and purification of the, e.g., microbially expressed antibodies or immunoglobulin chains of the invention may be by any conventional means such as, for example, preparative chromatographic separations and immunological separations such as those involving the use of monoclonal or polyclonal antibodies directed, e.g., against the constant region of the antibody of the invention. It will be apparent to those skilled in the art that the antibodies of the invention can be further coupled to other moieties for, e.g., drug targeting and imaging applications. Such coupling may be conducted chemically after expression of the antibody or antigen to site of attachment or the coupling product may be engineered into the antibody or antigen of the invention at the DNA level. The DNAs are then expressed in a suitable host system, and the expressed proteins are collected and renatured, if necessary.

The present invention also involves a method for producing cells capable of expressing an antibody of the invention or its corresponding immunoglobulin chain(s) comprising genetically engineering cells with the polynucleotide or with the vector of the invention. The cells obtainable by the method of the invention can be used, for example, to test the interaction of the antibody of the invention with its antigen. Furthermore, the invention relates to an antibody of the invention or fragment thereof encoded by a polynucleotide according to the invention or obtainable by the above-described methods or from cells produced by the method described above. The antibodies of the present invention will typically find use individually in treating substantially any disease susceptible to monoclonal antibody-based therapy. In particular, the immunoglobulins can be used for passive immunization or the removal of HCV or unwanted cells or antigens, such as by complement mediated lysis, all without substantial immune reactions (e.g., anaphylactic shock) associated with many prior antibodies. For an antibody of the invention, typical disease states suitable for treatment include chronic HCV infection.

In some embodiments, the antibodies of the present invention are used to quantify, localize, such as immunolocalize or in situ localize, or isolate a peptide of interest that is linked to the TAG. The antibodies of the invention are, for example, suited for use in immunoassays in which they can be utilized in liquid phase or bound to a solid phase carrier. Examples of immunoassays which can utilize the antigen of the invention are competitive and non-competitive immunoassays in either a direct or indirect format. Examples of such immunoassays are the radioimmunoassay (RIA), the sandwich (immunometric assay) and the Western blot assay. The antibodies of the invention can be bound to many different carriers and used to isolate peptides of interest linked to the TAG. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds; see also the embodiments discussed hereinabove.

The present invention also comprises methods of detecting the presence of TAG, or a peptide linked to the TAG, in a sample, comprising a sample, contacting said sample with one of the aforementioned antibodies, such as under non-reducing conditions permitting binding of the antibody to the TAG, and detecting the presence of the antibody so bound, for example, using immuno assay techniques such as radioimmunoassay or enzymeimmunoassay.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Generation of a Shuttle Vector for Gene Coexpression in Yeast

We generated a yeast shuttle vector pDRf1-GW-P_{HXT7} which contains a Gateway cloning cassette (Invitrogen, Carlsbad, Calif.) inserted between the PMA1 promoter (P_{PMA1}) and the ADH1 terminator (T_{ADH1}), and carries a second yeast expression cassette inserted into the SphI restriction site at the 3′-end of T_{ADH1}. This cassette contains the HXT7 promoter (P_{HXT7}) and the CYC1 terminator (T_{CYC1}), both separated by a multicloning site containing a NotI restriction site (P_{HXT7}-T_{CYC1}). The P_{HXT7}-T_{CYC1} and P_{PMA1}-T_{ADH1} expression cassettes are in the same orientation. To generate a pDRf1-GW-P_{HXT7} co-expression vector, the yeast shuttle vector p426 (Wieczorke et al. 1999) was first modified by site-directed mutagenesis (Kunkel 1985) to insert two SphI restriction sites at the 5′-end of P_{HXT7} and the 3′-end of T_{CYC1} using the following primers 5′-CGAAATTGTTCCTACGAGCTCGCATGCTTTTGTTCCCTTTAGTGAGG-3′ (SEQ ID NO:29) and 5′-GACTCACTATAGGGCGAATTGGCATGCGGCCGCAAATTAAAGCCTTC-3′ (SEQ ID NO:30), respectively. This vector was further modified to insert the unique NotI restriction site between P_{HXT7} and T_{CYC1}. The multi-cloning site and the sequence encoding a His-tag located between P_{HXT7} and T_{CYC1} was replaced by site-directed mutagenesis (Kunkel 1985) using the following primer 5′-CATAACTAATTACATGACTCGAGCGGCCGCCCGGGGGATCCACTAGA-3′ (SEQ ID NO:31). After mutagenesis, the P_{HXT7}-T_{CYC1} expression cassette was sequence-verified, digested with SphI (Fermentas Inc., Glen Burnie, Md.) and inserted into the unique SphI restriction site of pDRf1-GW located at the T_{ADH1} 3′-end (Logue et al. 2006).

Construction and Expression of Recombinant Yeast Harboring 4CL5 and HCBT

The 4CL5 gene (At3g21230) was cloned from Arabidopsis thaliana (ecotype Columbia). Four μg of total RNA was isolated from mixed organs of Arabidopsis plants using the RNeasy Plant Mini Kit (Qiagen, Valencia, Calif.) and used to perform an RT-PCR. First strand cDNAs were synthesized using the Transcriptor High Fidelity cDNA Synthesis kit (Roche, Indianapolis, Ind.) and used to amplify the 4CL5 gene using the following oligonucleotides containing NotI restriction sites: forward, 5′-GCGGCCGCATGGTGCTCCAACAACAAACGC-3′ (SEQ ID NO:32); and reverse, 5′-GCGGCCGCCTATTTAGAGCACATGGTTTCC-3′ (SEQ ID NO:33) (NotI sites are underlined). The PCR product was subcloned into the pCR-Blunt vector (Invitrogen, Carlsbad, Calif.), digested with NotI restriction enzyme (Fermentas Inc., Glen Burnie, Md.), gel purified, and ligated into the pDRf1-GW-pHXT7 vector at the unique NotI restriction site located between pHXT7 and tCYC1 of the expression cassette. A clone showing correct orientation for the 4CL5 gene was selected and the resulting vector was named pDRf1-4CL5-GW.

To clone the gene encoding HCBT, a gene sequence encoding the HCBT1 protein (O24645) without stop codon and flanked with the attB1 (5′-end) and attB2 (3′-end) Gateway recombination sites was synthesized and codon optimized for yeast expression by GenScript (Piscatway, N.J.). The attB1-HCBT-attB2 fragment was remobilized into the Donor plasmid vector pDONR221-f1 (Lalonde et al. 2010) by in-vitro BP recombination, and transferred into the pDRf1-4CL5-GW and pDRf1-GW-pHXT7 vectors by in-vitro LR recombination using the Gateway technology (Invitrogen, Carlsbad, Calif.). The resulting vectors were named pDRf1-4CL5-HCBT1 and pDRf1-HCBT1. A pDRf1-4CL5 control vector was also generated by in-vitro LR recombination between the pDRf1-4CL5-GW vector and an ENTRY clone containing only a nucleotide sequence corresponding to a PvuII restriction site between the attL recombination sites. This six-nucleotide sequence consequently replaced both the ccdB and chloramphenicol resistance genes of the Gateway cassette in the pDRf1-4CL5-GW vector.

pDRf1-4CL5-HCBT1, pDRf1-HCBT1 and pDRf1-4CL5 were transformed into the S. cerevisiae pad1 knockout (MATa his3Δ1 leu2Δ0 met15Δ0 ura3Δ0 Δpad1, ATCC 4005833; Winzeler et al. 1999) using the lithium acetate transformation method (Gietz and Woods 2002) and selected on solid medium containing Yeast Nitrogen Base (YNB) without amino acids (Difco 291940; Difco, Detroit, Mich.) supplemented with 3% glucose and 1× dropout-uracil (CSM-ura; Sunrise Science Products, San Diego, Calif.).

### HCBT Expression Analysis

The codon optimized HCBT clone was synthesized without a stop codon, therefore generating an in-frame C-terminal tag corresponding to the PAFLYKVV (SEQ ID NO:9) peptide after translation of the attB2 site obtained after LR recombination. A polyclonal antibody was raised against an AttB2 peptide (DPAFLYKVVD (SEQ ID NO:3)) using rabbit as a host, and purified using an affinity column (Biogenes, Berlin, Germany). The purified serum was named ‘universal antibody’ since it can be used to quantify the expression level of any protein expressed with any Gateway destination vectors.

For soluble protein extraction, overnight cultures from single colonies were used to inoculated 50 ml of 2× yeast nitrogen base medium without amino acids (Difco, Detroit, Mich.) supplemented with 6% glucose and 2×CSM-Ura (Sunrise Science Products, San Diego, Calif.) at an OD₆₀₀=0.15, and incubated at 30° C. until it reached OD₆₀₀=1. Cells were centrifuged at 4500×g for 5 min at 4° C. and washed with one volume of chilled-water. The cell pellets were resuspended in 300 μL of CelLytic-Y yeast cell lysis/extraction reagent (Sigma-Aldrich, St. Louis, Mo.) supplemented with 10 mM dithiothreitol, 2 mM phenylmethanesulfonylfluoride, and 2% protease inhibitor cocktail (v/v, P8215 Sigma, St. Louis, Mo.). Approximately 200 μL of acid-washed glass beads (Sigma, St. Louis, Mo.) were added to the mixture, which was then vortexed ten times for 30 sec, and centrifuged at 10,000×g for 5 min at 4° C. to collect the supernatant. Samples were maintained on ice between vortexing steps. The supernatant containing soluble proteins was collected and used for immunoblotting.

Protein concentration was quantified using the Bradford method (Bradford 1976) and bovine serum albumin as a standard. For electrophoresis, soluble protein (5 μg) were mixed with 0.2 M Tris-HCl, pH 6.5, 8% (w/v) SDS, 8% (v/v) (3-mercaptoethanol, 40% (v/v) glycerol, and 0.04% (w/v) bromophenol blue and incubated at 40° C. for 30 min. Proteins were separated by SDS-PAGE using 8-16% (w/v) polyacrylamide gradient gels (Invitrogen, Carlsbad, Calif.) and electrotransferred (100 volts, 45 min) onto PVDF membranes (Thermo Fisher Scientific, Rockford, Ill.). Blotted membranes were incubated 1 h in TBS-T (20 mM Tris-HCl, 150 mM NaCl, 0.1% (v/v) Tween 20, pH 7.6) containing 2% (w/v) non-fat milk powder, and incubated overnight with the universal antibody (1:20000) in TBS-T containing 2% (w/v) non-fat milk powder. Membranes were then washed in TBS-T for 30 min and incubated for 1 h with an anti-rabbit secondary antibody conjugated to horseradish peroxidase (1:20000; Sigma-Aldrich, St. Louis, Mo.) in TBS-T containing 2% (w/v) non-fat milk powder. Membranes were then washed in TBS-T for 30 min, and detection was performed by chemiluminescence using the SuperSignal West Dura Extended Duration Substrate (Thermo Fisher Scientific, Rockford, Ill.).

### Production of Cinnamoyl Anthranilates

An overnight culture from a single colony of the pDRf1-4CL5-HCBT recombinant yeast grown on 2×YNB medium without amino acids supplemented with 6% glucose and 2×CSM-Ura was used to inoculated 15 mL of fresh minimal medium at an OD₆₀₀=0.15 and shaken at 200 rpm in a 30° C. room. When the 10-mL culture reached an OD₆₀₀=1, all substrates were added at once to reach final concentrations of 500 μM for anthranilate and 3-hydroxyanthranilate, and 300 μM for the cinnamic acids except for 3-methoxycinnamic acid, 4-methoxycinnamic acid, and 2,5-dimethoxycinnamic acid which were supplied at a final concentration of 50 μM due to their negative effect on cell growth at higher concentrations. The cultures were shaken at 200 rpm in a 30° C. room for 15 h for the production of cinnamoyl anthranilates. As negative controls, yeast colonies harboring the pDRf1-HCBT1 or pDRf1-4CL5 vectors were grown using similar conditions.

### Expression Analysis of the HCBT Enzyme in Recombinant Yeast

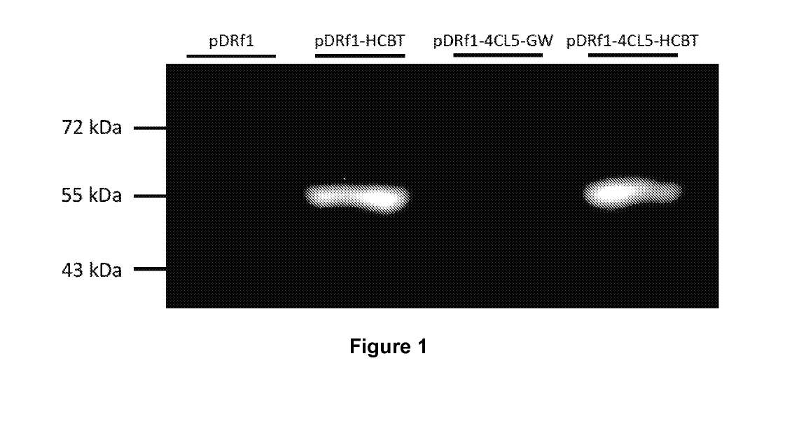

To verify HCBT expression, we conducted immunoblotting analysis on crude protein extracts obtained from recombinant yeast strains harboring pDRf1-HCBT and pDRf1-4CL5-HCBT, respectively. As shown in FIG. 1, a specific signal corresponding to an approximately 53-kDa protein was detected only in protein extracts derived from the yeast strain harboring the HCBT gene, which is in accordance with the predicted size of HCBT tagged with the AttB2 peptide.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An antibody or fragment thereof capable of specifically binding to an epitope of the amino acid sequence CDPAFLYKVVD (SEQ ID NO:1) or a fragment of at least 5 amino acids thereof.

2. The antibody or fragment thereof of claim 1, wherein the epitope is presented by an amino acid sequence selected from the group consisting of CDPAFLYKVV (SEQ ID NO:2), DPAFLYKVVD (SEQ ID NO:3), CDPAFLYKV (SEQ ID NO:4), DPAFLYKVV (SEQ ID NO:5), PAFLYKVVD (SEQ ID NO:6), CDPAFLYK (SEQ ID NO:7), DPAFLYKV (SEQ ID NO:8), PAFLYKVV (SEQ ID NO:9), FLYKVVD (SEQ ID NO:10), CDPAFLY (SEQ ID NO:11), DPAFLYK (SEQ ID NO:12), PAFLYKV (SEQ ID NO:13), AFLYKVV (SEQ ID NO:14), FLYKVVD (SEQ ID NO:15), CDPAFL (SEQ ID NO:16), DPAFLY (SEQ ID NO:17), PAFLYK (SEQ ID NO:18), AFLYKV (SEQ ID NO:19), FLYKVV (SEQ ID NO:20), LYKVVD (SEQ ID NO:21), CDPAF (SEQ ID NO:22), DPAFL (SEQ ID NO:23), PAFLY (SEQ ID NO:24), AFLYK (SEQ ID NO:25), FLYKV (SEQ ID NO:26), LYKVV (SEQ ID NO:27), and YKVVD (SEQ ID NO:28).

3. A polynucleotide encoding the antibody or fragment thereof of claim 1.

4. A vector comprising the polynucleotide of claim 3.

5. A cell comprising the polynucleotide of claim 3.

6. A hybridoma capable of producing an antibody or fragment thereof of claim 1.

7. A method of isolating a peptide of interest, comprising:
(a) contacting (i) a peptide of interest linked to the amino acid sequence CDPAFLYKVVD (SEQ ID NO:1) or a fragment thereof, and (ii) the antibody or fragment thereof of claim 1, and
(b) separating at least a partial population of the antibody or fragment thereof, and any bound molecule thereto, from molecules not bound to the antibody or fragment thereof.

8. The method of claim 7, wherein the contacting step comprises introducing a first solution comprising the peptide of interest linked to the amino acid sequence CDPAFLYKVVD (SEQ ID NO:1) or a fragment thereof, and a second solution comprising the antibody or fragment thereof.

9. The method of claim 7, further comprising linking the peptide of interest to the amino acid sequence CDPAFLYKVVD (SEQ ID NO:1) or a fragment thereof.

10. The method of claim 7, further comprising expressing the peptide of interest linked to the amino acid sequence CDPAFLYKVVD (SEQ ID NO:1) or a fragment thereof in a host cell, or in vitro in a reaction solution, comprising a polynucleotide encoding peptide of interest linked to the amino acid sequence CDPAFLYKVVD (SEQ ID NO:1) or a fragment thereof.

11. The method of claim 10, further comprising linking a first polynucleotide encoding the peptide of interest and second polynucleotide encoding the amino acid sequence CDPAFLYKVVD (SEQ ID NO:1) or a fragment thereof.

12. A kit comprising: a vector comprising a nucleotide sequence encoding the amino acid sequence CDPAFLYKVVD (SEQ ID NO:1) or a fragment thereof linked to one or more restriction sites, and an antibody or fragment thereof capable of specifically binding to an epitope of the amino acid sequence CDPAFLYKVVD (SEQ ID NO:1) or a fragment thereof.
